(19) United States
(12) Patent Application Publication
(10) Pub. No.: US 2018/0343878 A1
(43) Pub. Date: Dec. 6, 2018

(54) METHODS FOR REDUCING CONTAMINANTS IN AGRICULTURAL COMMODITIES WITH HUMID OZONE

(71) Applicant: Archer Daniels Midland Company, Decatur, IL (US)

(72) Inventors: Perry Doane, Decatur, IN (US); Aaron Johnson, Pocatello, ID (US)

(21) Appl. No.: 15/757,522

(22) PCT Filed: Sep. 8, 2016
(86) PCT No.: PCT/US16/50777

§ 371 Date: Mar. 5, 2018

Related U.S. Application Data
(60) Provisional application No. 62/215,461, filed on Sep. 8, 2015.

(51) Int. Cl. A23B 9/18

(52) U.S. Cl. CPC A23B 9/18

(57) ABSTRACT

Improved methods for treating agricultural commodities with ozone are disclosed. The methods include treating the commodity with humid ozone to effectively treat the commodity.

### CROSS-REFERENCE TO RELATED APPLICATIONS

This application claims priority to U.S. Provisional Patent Application No. 62/215,461, filed Sep. 8, 2015, the contents of the entirety of which is incorporated by this reference.

### TECHNICAL FIELD

The present relates generally to ozone treatment of agricultural commodities, and more particularly to treating such agricultural commodities with humid ozone.

### BACKGROUND OF THE INVENTION

Ozone can be used to treat agricultural commodities for various things such as insects, mold, bacteria, unwanted odors, unwanted chemical compounds, and/or toxins. The ozone can be introduced into a storage container in order to treat the commodity being stored therein.

Ozonation is allowed as a treatment for grains used in human foods and is effective to diminish the microbial load for whole grains. There is also evidence to suggest that ozone is able to effectively reduce other contaminants in agricultural commodities such as mycotoxins and/or unwanted chemical compounds.

However, while ozone is known to reduce various contaminants, needs exist to more effective methods of treating various commodities with ozone.

### SUMMARY OF THE INVENTION

In each of its various embodiments, the present invention solves these challenges and discloses improved methods of treating grain with humid ozone. As ozone generators typically produce ozone in conditions that are essentially devoid of moisture, it was unexpectedly discovered by the inventors that humid ozone is able to more effectively reduce contaminants in agricultural commodities as compared to dry ozone.

In one embodiment, a method of treating an agricultural commodity with humid ozone comprises placing the agricultural commodity in contact with an effective amount of the humid ozone, thus reducing a level of at least one contaminant in the agricultural commodity.

In a further embodiment, a system for reducing contaminants in an agricultural commodity comprises a first portion of the agricultural commodity having a contaminant; means for reducing the contaminants; and a second portion of the agricultural commodity having a reduced amount of contaminants.

### DESCRIPTION OF THE DRAWINGS

The characteristics and advantages of the present disclosure may be better understood by reference to the accompanying figures, in which:

FIG. 1 shows one embodiment of a system for treating an agricultural commodity of the present invention with ozone.

FIG. 2 illustrates the ability of one embodiment of the present invention to reduce contaminants in an agricultural commodity.

FIG. 3 illustrates the ability of another embodiment of the present invention to reduce contaminants in an agricultural commodity.

### DETAILED DESCRIPTION OF THE INVENTION

Work on treating agricultural commodities with ozone has continued. The following patent applications are assigned to the assignee of the present invention and describe general methods and conditions for treating grain with ozone. US Patent Application Publication 2011/0151079, the contents of the entirety of which are incorporated by this reference in its entirety, describes a continuous treatment of grain with ozone, US Patent Application Publication 2011/0151080, the contents of the entirety of which are incorporated by this reference in its entirety, described downdraft methods of treating grain with ozone, and US Patent Application Publication 2011/0151073, the contents of the entirety of which are incorporated by this reference in its entirety, describes treating grain with ozone in grain piles.

Agricultural commodities which may be treated according to the methods of the present invention include, but are not limited to, any agricultural commodity that is typically stored in piles or in bins such as wheat, corn, soybeans, barley, oats, rye, rice, millet, sorghum, sunflowers, flax, canola, triticale, cocoa beans, quinoa, amaranth, buckwheat, chia, hemp, milo, or other grains. Other agricultural commodities or products that may be treated with methods of the present invention include, but are not limited to dried fruits, coconut, nuts, almonds, brazil nuts, cashews, filberts, macadamias, pecans, pinenuts, pistachios, peanuts, walnuts, legumes, edible beans, chickpeas, kidney beans, lentils, lima beans, navy beans, pinto beans, seeds, caraway, pumpkins seeds, and/or sunflower seeds.

Treatment of the agricultural commodity with the ozone reduces a contaminant located within the agricultural commodity. Contaminants that may be removed from the agricultural commodity includes, but are not limited to, smoky odors, off odors, polycyclic aromatic hydrocarbons, microbes, molds, fungi, bacteria, Salmonella, E. coli, mycotoxins, vomatoxins, or other mold toxins from the agricultural commodity.

The effective amount of ozone may be at least 1,000 ppm of the ozone, at least 2,000 ppm of the ozone, at least 3,000 ppm of the ozone, at least 4,000 ppm of the ozone, at least 5,000 ppm of the ozone, between 1,000 and 10,000 ppm of the ozone, between 2,000 and 10,000 ppm of the ozone, between 4,000 and 10,000 ppm of the ozone, between 1,000 and 12,000 ppm of the ozone, between 2,000 and 12,000 ppm of the ozone, or between 4,000 and 12,000 ppm of the ozone. The effective amount of ozone may also be expressed in grams (g) of ozone per kilograms (kg) of agricultural commodity treated. The effect amount may be at least 1 g/kg, at least 2 g/kg, at least 3 g/kg, at least 4 g/kg, at least 5 g/kg, at least 6 g/kg, at least 7 g/kg, at least 8 g/kg, at least 9 g/kg, at least 10 g/kg, up to 15 g/kg, up to 26 g/kg, up to 30 g/kg, between 1-30 g/kg (including ranges including all integers between), between 2-25 g/kg, between 3-25 g/kg, between 3-25 g/kg, between 4-25 g/kg, or between 5-25 g/kg.

The ozone may be placed in contact with the agricultural commodity for an effective amount of time necessary to reduce a level of contaminants. The necessary amount of time may be at least 2 hours, at least 4 hours, at least 6 hours, between 4-55 hours, or between 6-55 hours.

The ozone may be placed in contact with the agricultural commodity at a relative humidity of at least 50%, at least 60%, at least 70%, at least 80%, at least 90%, between 50-90%, between 60-90%, between 70-90%, or between 80-90%.

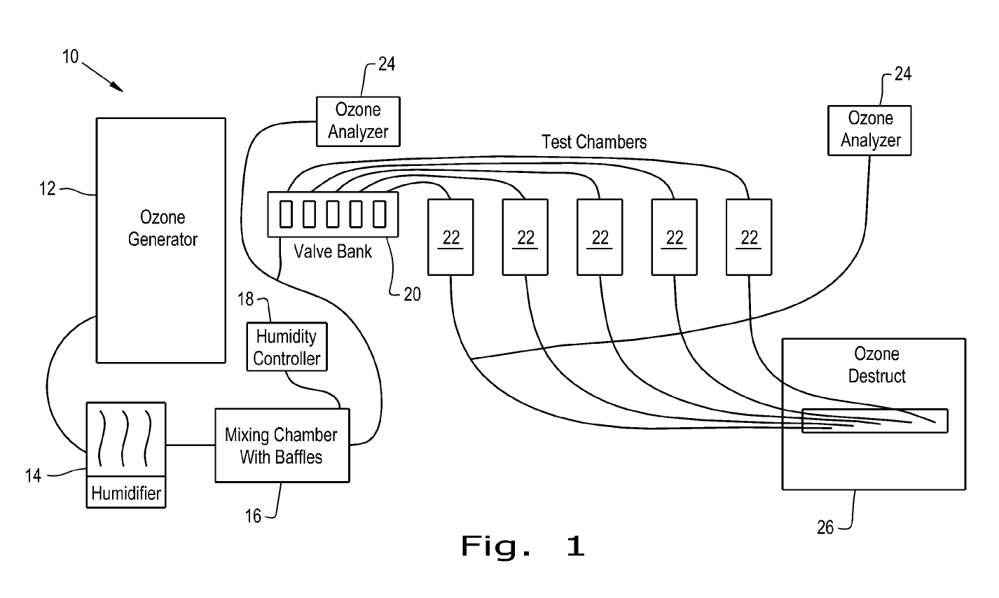

One embodiment of a system 10 that may be used to treat an agricultural commodity with ozone is shown generally in FIG. 1. The system 10 includes an ozone generator 12. The ozone produced in the ozone generator 12 travels through a humidifier 14 and a mixing chamber 16 that functions in concert with a humidity controller 18 to achieve the desired relative humidity of the ozone. The humid ozone may travel through a valve bank 20 and into at least one ozone treatment chamber 22, where the valve bank 20 may be used to control to which ozone treatment chamber 22 the humid ozone travels. Ozone analyzers 24 may be located within the system 10 to analyze the amount of ozone in the humid ozone that is traveling through the system. Once the humid ozone passes through the ozone treatment chamber 22, the humid ozone may enter an ozone destruct chamber 26 which may be used to destroy, treat, or possibly recycle the ozone back into the system 10.

### EXAMPLES

The present invention is further demonstrated by the examples that follow. Those having ordinary skill in the art will appreciate that variations of these Examples are possible within the scope of the invention.

### Example 1

Examples 2, 4, and 5 of PCT patent application no. PCT/US15/48855, filed Sep. 8, 2015 and entitled Methods of Reducing Contaminants in Cocoa Beans (the contents of the entirety of which is incorporated by this reference), assigned to Archer-Daniels-Midland Company, shows the ability of humid ozone to reduce contaminants in cocoa beans. Example 2 demonstrates how humid ozone can reduce smoky odors in cocoa beans, Example 4 demonstrates how humid ozone can reduce polycyclic aromatic hydrocarbons (PAHs) in cocoa beans more effectively than dry ozone, and Example 5 demonstrates that humid ozone reduces cocoa bean odors, microbial counts, and PAH contents in products of the cocoa beans.

### Example 2

Two lots of wheat having a range of vomitoxin contamination (i.e., 3-6 ppm and 8-10 ppm, respectively) were obtained. Wheat samples from the two lots were exposed to ozone for 12 or 24 hours at two (2) concentrations of ozone, 4 & 16 g/M⁻³ to obtain a range of total ozone exposure (about 12.5 to 100 g O₃/kg wheat). The air used to deliver the ozone was adjusted to 35% or 70% relative humidity. This example was a partially replicated randomized design. In this example, each ozone chamber was loaded with 1 kg of wheat for evaluation.

After the ozone treatments, the treated wheat was divided and samples of the treated wheat were measured for vomitoxin according to known vomitoxin assays. Microbial analysis vomitoxin was also determined for all samples. The equipment and design of a system used to delivery humid ozone described herein with reference to FIG. 1 was used to treat the wheat.

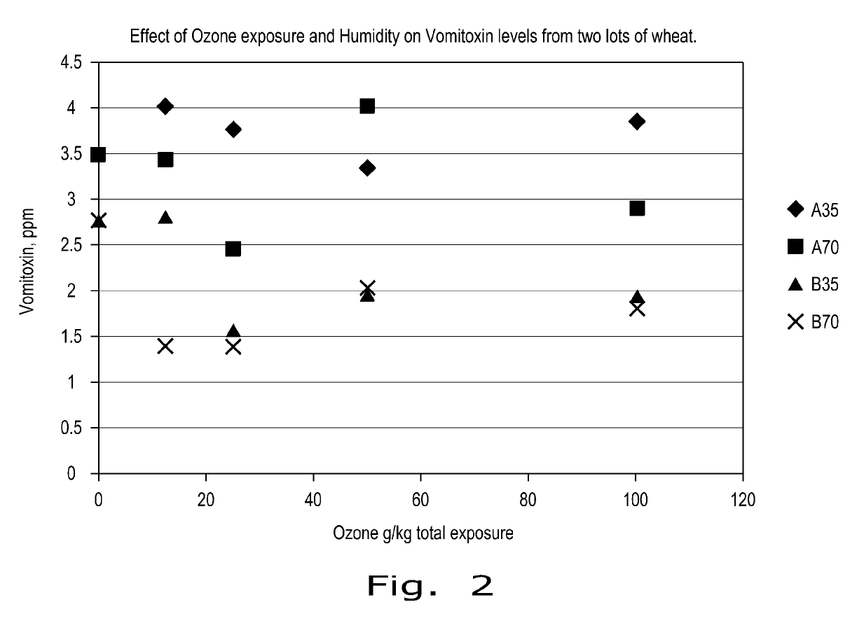

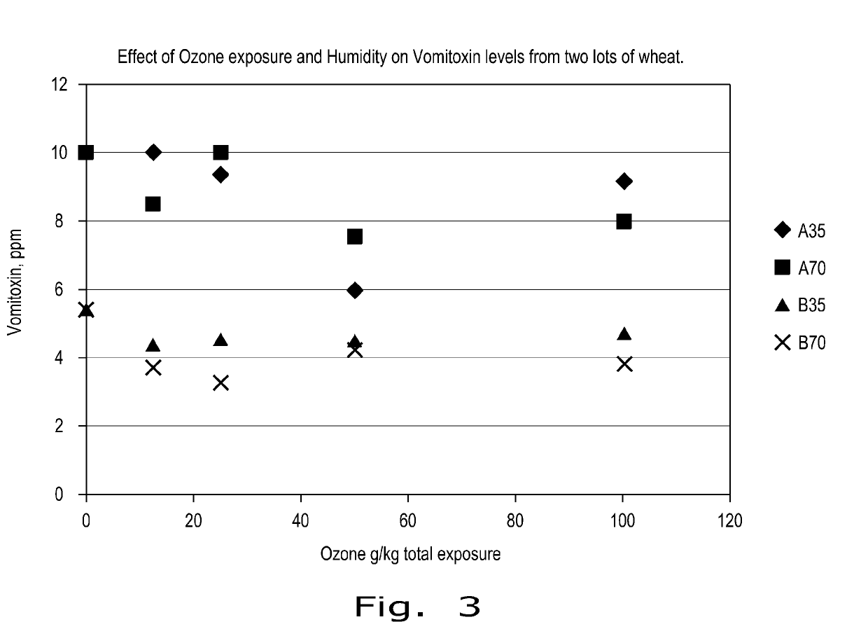

The humid ozone reduced microbial count, yeast, and molds as shown in Table 1. The ozonation also reduced vomitoxin (see, Table 1) and this effect was improved in the presence of the elevated humidity. The decrease in vomitoxin appears greater or to some extent more affected by humidity in the wheat with greater vomitoxin contamination as demonstrated by measuring the vomitoxin two different times as shown in FIG. 2 (measurement 1, msmt 1) and FIG. 3 (measurement 2, msmt 2). It also appeared that the duration of ozone exposure (total exposure) did improve response and supports some “dwell time” for ozonation in wheat storage. Table 1 shows the results of these analysis.

TABLE 1
Table 1. Effect of Humidity Level and Ozone Concentration
on Microbial count and Vomitoxin levels in stored wheat.

| | Vomitoxin | | Microbial Analysis | |
|---|---|---|---|---|
| | Msmt 1 | Msmt 2 | SPC | YM |
| Lot A | 3.49 | 10 | 3800 | 127 |
| Lot B | 2.77 | 5.4 | 5733 | 343 |
| Ozone, | | | | |
| g M⁻³ | | | | |
| 0 | 3.13 | 7.71 | 4766.7 | 235.0 |
| 4 | 2.87 | 6.11 | 612.5 | 12.9 |
| 16 | 2.46 | 6.61 | 575 | 11.1 |
| Relative | | | | |
| Humidity | | | | |
| 0 | 3.13 | 7.71 | 4766.7 | 235.0 |
| 35 | 2.91 | 6.59 | 725 | 13.3 |
| 70 | 2.43 | 6.14 | 462.5 | 10.8 |
| Exposure | | | | |
| Time, h | | | | |
| 0 | 3.13 | 7.71 | 4766.67 | 235 |
| 12 | 2.6 | 6.72 | 762.5 | 12.9 |
| 24 | 2.73 | 6 | 425 | 11.1 |
| O3:RH0 | | | | |
| 0 | 3.13 | 7.71 | 4766.7 | 235 |
| 4:35 | 3.03 | 6.22 | 725 | 17.5 |
| 4:70 | 2.72 | 6.00 | 500 | 8.25 |
| 16:35 | 2.78 | 6.95 | 725 | 9.00 |
| 16:70 | 2.14 | 6.27 | 425 | 13.2 |

Tempered wheat was also treated with humid ozone. The tempering results are presented in Table 2. Each wheat lot was subsampled and 1 kg samples were placed in a paint shaker. Water was added to obtain an estimated 16% moisture, and the sample was mixed, and shaken for 5 minutes. After adding moisture, samples were placed in the chambers and ozonated at 35% relative humidity as described in this Example. The use of ozone in this tempering system reduced vomitoxin with some samples reaching the target of 2 ppm. Time did not appear to be a significant effect, although numerically longer exposure provided the lowest values. Increased concentration of ozone was also associated with the lower mean vomitoxin results. This may suggest that initial response is rapid, but that across more samples extent of vomitoxin reduction can respond to total exposure.

TABLE 2
Table 2. Effect of Ozone Concentration and Tempering time
on Vomitoxin levels in wheat. (2 different measurements)

| | Vomitoxin | |
|---|---|---|
| | Msmt 1 | Msmt 2 |
| O3 | | |
| 0 | 4.05 | 7.95 |
| 4 | 2.15 | 6.05 |
| 16 | 1.89 | 4.88 |
| Time, h | | |
| 0 | 4.05 | 7.95 |
| 8 | 2.17 | 5.34 |
| 12 | 1.88 | 5.41 |
| 16 | 2.01 | 5.65 |
| O3:Time | | |
| 0 | 4.05 | 7.95 |
| 4:8 | 2.21 | 5.68 |
| 4:12 | 1.99 | 6.31 |
| 4:16 | 2.25 | 6.17 |
| 16:8 | 2.14 | 5 |
| 16:12 | 1.76 | 4.52 |
| 16:16 | 1.78 | 5.12 |

Table 3 provides data for this Example showing the effect of humidity level and ozone concentration of the humid ozone on lots of stored wheat.

TABLE 3
Effect of Humidity Level and Ozone Concentration on Microbial
count and Vomitoxin levels for Individual Lots of in stored wheat.

| Relative | Ozone | Exposure | Standard | Yeast and | Vomitoxin | |
|---|---|---|---|---|---|---|
| Humidity | gM⁻³ | time, h | Plate count | Mold | Measurement 1 | Msmt 2 |

Wheat Lot A

| | | | | | | |
|---|---|---|---|---|---|---|
| Control | Control | Control | 3,800 | 127 | 3 | 10 |
| 35 | 4 | 12 | 200 | 10 | 4.01 | 10.00 |
| 35 | 4 | 24 | 400 | 40 | 3.34 | 5.97 |
| 35 | 16 | 12 | 1500 | 3 | 3.76 | 9.36 |
| 35 | 16 | 24 | 200 | 3 | 3.85 | 9.16 |
| 70 | 4 | 12 | 500 | 10 | 3.43 | 8.49 |
| 70 | 4 | 24 | 100 | 10 | 4.02 | 7.55 |
| 70 | 16 | 12 | 200 | 20 | 2.46 | 10.00 |
| 70 | 16 | 24 | 300 | 3 | 2.90 | 7.98 |

Wheat Lot B

| | | | | | | |
|---|---|---|---|---|---|---|
| Control | | | 5,733 | 343 | 3 | 5 |
| 35 | 4 | 12 | 1,200 | 10 | 2.81 | 4.39 |
| 35 | 4 | 24 | 1,100 | 10 | 1.96 | 4.52 |
| 35 | 16 | 12 | 600 | 20 | 1.57 | 4.55 |
| 35 | 16 | 24 | 600 | 10 | 1.95 | 4.73 |
| 70 | 4 | 12 | 1,200 | 10 | 1.39 | 3.72 |
| 70 | 4 | 24 | 200 | 3 | 2.03 | 4.24 |
| 70 | 16 | 12 | 700 | 20 | 1.38 | 3.28 |
| 70 | 16 | 24 | 500 | 10 | 1.80 | 3.82 |

Table 4 provides data for this Example showing the effect of humidity level and ozone concentration of the humid ozone on lots of stored wheat.

TABLE 4
Effect of Ozone and Tempering time on
Vomitoxin levels in Two Lots of wheat.

| | |
|---|---|
| Wheat Lot A | Wheat Lot B |

| | | | | |
|---|---|---|---|---|
| Temper | 4 gM⁻³ | 16 gM⁻³ | 4 gM⁻³ | 16 gM⁻³ |
| Time | O₃ | O₃ | O₃ | O₃ |

Vomitoxin - Measurement 1 results

| | | | | |
|---|---|---|---|---|
| Control | 10.00 | 10.00 | 5.90 | 5.90 |
| 8 h | 9.06 | 7.83 | 2.46 | 1.97 |
| 8 h | 8.98 | 7.61 | 2.20 | 2.59 |
| 12 h | 9.17 | 6.91 | 3.96 | 2.74 |
| 12 h | 10.00 | 6.61 | 2.09 | 1.82 |
| 16 h | 8.53 | 7.21 | 3.45 | 3.20 |
| 16 h | 8.70 | 7.78 | 3.99 | 2.30 |

Vomitoxin - Measurement 2 results

| | | | | |
|---|---|---|---|---|
| Control | 5.44 | 5.44 | 2.66 | 2.66 |
| 8 h | 2.87 | 3.27 | 1.41 | 1.00 |
| 8 h | 3.11 | 2.85 | 1.45 | 1.42 |
| 12 h | 2.43 | 2.52 | 1.58 | 1.54 |
| 12 h | 2.47 | 1.96 | 1.48 | 1.05 |
| 16 h | 3.73 | 2.26 | 1.47 | 1.20 |
| 16 h | 2.48 | 2.15 | 1.33 | 1.51 |

While this invention has been particularly shown and described with references to exemplary embodiments thereof, it will be understood by those skilled in the art that various changes in form and details may be made therein without departing from the scope of the invention encompassed by the appended claims.

1. A method of treating an agricultural commodity with humid ozone, the method comprising:
placing the agricultural commodity in contact with an effective amount of the humid ozone, thus reducing a level of at least one contaminant in the agricultural commodity.

2. The method of claim 1, wherein the humid ozone has a relative humidity of at least 50%.

3. The method of claim 1, wherein the contaminant is selected from the group consisting of a smoky odor, an off odor, a microbe, a polycyclic aromatic hydrocarbon, a mycotoxin, a vomatoxin, and combinations of any thereof.

4. The method of claim 1, wherein the effective amount of ozone is at least 1,000 ppm.

5. The method of claim 1, wherein the effective amount of ozone ranges from 1,000-12,000 ppm or between 1-30 grams of ozone per kilogram of the agricultural commodity.

6. The method of claim 1, wherein the agricultural commodity is in contact with the effective amount of ozone for at least 4 hours.

7. The method of claim 1, wherein the agricultural commodity is placed into contact with the effective amount of ozone at a relative humidity of between 50-90%.

8. The method of claim 1, further comprising:
placing the agricultural commodity having in a container; and
introducing the effective amount of the humid ozone into the container, thus placing the agricultural commodity in contact with the effective amount of the humid ozone.

9. The method of claim 1, further comprising passing the effective amount of the humid ozone through the agricultural commodity with an air flow.

10. The method of claim 9, wherein the air flow ranges from 30-70 liters/minute.

11. The method of claim 1, wherein passing the effective amount of the humid ozone through the agricultural commodity comprises an act selected from the group consisting of creating negative pressure in the container such that the ozone flows through the agricultural commodity, creating positive pressure in the container with the ozone such that the ozone flows through the agricultural commodity, or a combination thereof.

12. The method of claim 1, wherein the method is a batch process.

13. The method of claim 1, wherein the method is a continuous process.

14. The method of claim 1, wherein the agricultural commodity is selected from the group consisting of wheat, corn, soybeans, barley, oats, rye, rice, millet, sorghum, sunflowers, flax, canola, triticale, cocoa beans, quinoa, amaranth, buckwheat, chia, hemp, milo, dried fruits, coconut, nuts, almonds, brazil nuts, cashews, filberts, macadamias, pecans, pinenuts, pistachios, peanuts, walnuts, legumes, edible beans, chickpeas, kidney beans, lentils, lima beans, navy beans, pinto beans, seeds, caraway, pumpkins seeds, sunflower seeds, and combinations of any thereof.

15. The method of claim 1, further comprising an act selected from the group consisting of passing ozone over water to increase the humidity of the ozone; evaporating water and combining the evaporated water with the ozone; and combinations thereof.

16. A system for reducing contaminants in an agricultural commodity, comprising:
a first portion of the agricultural commodity having a contaminant;
means for reducing the contaminants; and
a second portion of the agricultural commodity having a reduced amount of contaminants.

17. The system of claim 16, wherein the means for reducing the contaminants comprises:
humid ozone; and
a device for placing the humid ozone in contact with the agricultural commodity.

18. The system of claim 16, further comprising a third portion of the agricultural commodity in contact with an effective amount of the humid ozone that reduces the level of contaminants in the agricultural commodity.

19. The system of claim 16, further comprising a device for increasing the relative humidity of the ozone.

20. The system of claim 16, wherein the agricultural commodity is selected from the group consisting of wheat, corn, soybeans, barley, oats, rye, rice, millet, sorghum, sunflowers, flax, canola, triticale, cocoa beans, quinoa, amaranth, buckwheat, chia, hemp, milo, dried fruits, coconut, nuts, almonds, brazil nuts, cashews, filberts, macadamias, pecans, pinenuts, pistachios, peanuts, walnuts, legumes, edible beans, chickpeas, kidney beans, lentils, lima beans, navy beans, pinto beans, seeds, caraway, pumpkins seeds, sunflower seeds, and combinations of any thereof.
